(19) United States
(12) Patent Application Publication
(10) Pub. No.: US 2022/0042984 A1
(43) Pub. Date: Feb. 10, 2022

(54) Lateral Flow Assay Device for Detection of Analytes and Method of Detection Thereof

(71) Applicant: Rajanikanth Vangala, BANGALORE (IN)

(72) Inventor: Rajanikanth Vangala, BANGALORE (IN)

(21) Appl. No.: 17/394,380

(22) Filed: Aug. 4, 2021

Related U.S. Application Data
(60) Provisional application No. 62/706,205, filed on Aug. 5, 2020.

(51) Int. Cl. G01N 33/543; G01N 33/569

(52) U.S. Cl. CPC G01N 33/54388; G01N 33/54346; G01N 2800/26; G01N 2333/165; G01N 33/56983

(57) ABSTRACT

The present invention relates to a lateral flow assay device for detection of an analyte in a sample and a method of detection thereof. The present invention provides a quantitative assay for detection of an analyte in a sample. The present invention also provides a conjugate. The present invention provides a method of diagnosing COVID 19 in a patient.

### PRIORITY

This application claims the benefit under 35 U.S.C. § 119 of U.S. Provisional Application No. 62/706,205, filed on Aug. 5, 2020, and entitled “BIOMOLECULE/NANOPARTICLE SENSOR FOR DETECTION OF ANALYTES,” which is incorporated herein by reference in its entirety.

### FIELD OF THE INVENTION

The present invention relates to a lateral flow assay device for detection of an analyte in a sample and a method of detection thereof.

### BACKGROUND OF THE INVENTION

Early identification and detection of virus as cause of illness is important in order to prescribe an effective mode of treatment especially for high risk groups such as person suffering from co-morbidities, pregnant women, immunocompromised individuals etc.,

Proper and accurate diagnosis ensures that proper monitoring takes place and adequate control measures are instituted especially during an epidemic or a pandemic to prevent further spread of disease in the population.

Present analytical biosensors require specimens to be processed and require a capture antibody to bind to specific analyte in a specimen followed by addition of another detection antibody to detect the analyte. Other systems use chemiluminescence or calorimetric detection of an analyte using antibody conjugated to horseradish peroxidase enzyme (HRP) or biotin, for example. Conventional techniques for protein analytes such as enzyme-linked immunosorbent assay (ELISA) are time consuming and expensive. Other label free technologies (e.g., without radioactive, chromogenic, fluorescent etc. labels) based on surface plasmon resonance (SPR) and piezoelectric devices are usually rapid but require costly and high-end infrastructure with complex detection technologies. Recently, the colorimetric change due to aggregation of gold nanoparticles has been developed. The aggregation of gold nanoparticles happens in a controlled fashion and was used as a sensor by Mirkin et al, using DNA hybridization to induce an assembly of particles conjugated with single stranded DNA. See, for example Storhoff et al., “One-Pot colorimetric Differentiation of Polynucleotides with single base imperfections using gold nanoparticle probes”, 1998 Journal of The American Chemical Society, 120, pages 1959-1964. As can be seen, there is a need for rapid, simple, specific, and inexpensive bioassays and sensors. Gold nanoparticles have a high extinction coefficient due to plasmonic properties of the particles. Aggregation of gold nanoparticles causes a big shift in the extinction spectrum of suspensions shown as a colour change from red to purple, thus making gold nanoparticles suitable as a simple sensor to detect analytes. However, larger nanoparticles from 20 to 100 nm already have colour range from light purple to dark purple and may not show a colour change when used in detecting systems.

All present lateral flow assay systems use large size gold nanoparticles ranging from 40 to 100 nm in size which are already purple due to the size. This is due to the fact that they use antibodies as biosensors and antibodies are large is size with average molecular weight of 150 Kilo Daltons. The use of antibodies conjugated to large gold nanoparticles give colour when bind to analyte. However, this is not colour change from red to purple and are not sensitive enough. Average sensitivity of lateral flow assays is ˜65-70%. Therefore, there is need to develop methods of using gold nanoparticles with biosensors which can produce much more sensitive lateral flow or other quantitative colorimetric assays.

### SUMMARY OF THE INVENTION

The present invention relates to a lateral flow assay device (100) comprising porous membrane (20) mounted on a solid support (10). The said porous membrane have a sample pad (14) for receiving a liquid sample (12) comprising a target analyte at a first end and an absorbent pad (28) at the second end. The said solid support (10) permits capillary flow of the liquid sample comprising the target analyte from the sample pad (14) to the absorbent pad (28). It is characterized in that the porous membrane (20) comprises a conjugate pad (16) comprising gold nanoparticle sensor conjugate (18). The said gold nanoparticle sensor conjugate comprises gold nanoparticles having a particle size of 10 nm to 20 nm conjugated with a peptide that specifically binds with a protein in the target analyte or said gold nanoparticle sensor conjugate comprises gold nanoparticles having a particle size of 10 nm to 20 nm conjugated with an antibody against the protein in the target analyte. A test region (22) comprising an immobilized capture molecule (24) is provided. The said capture molecule is a peptide capable of specifically binding to the protein in the target analyte or an antibody against protein in the target analyte. Optionally a control region (26) comprising the protein in the target analyte immobilized on the porous membrane (20) is provided.

The gold nanoparticle is conjugated with the peptide capable of specifically binding with the protein in the liquid sample (12) and the capture molecule comprises the immobilized antibody against the protein in the liquid sample (12).

The gold nanoparticle is conjugated with the antibody against the protein in the liquid sample (12) and the capture molecule comprises the peptide capable of specifically binding with the protein in the liquid sample (12).

The gold nanoparticle is conjugated with the peptide capable of specifically binding with a spike protein or protein of the target analyte or with the antibody against the nucleocapsid protein of the target analyte. The gold nanoparticles comprise 30 μg-50 μg of the peptide or 0.5 μg-1 μg of the antibody.

The present invention provides a lateral flow assay device (100) where the capture molecule (24) is the peptide capable of specifically binding with a spike protein or a protein of the target analyte or the antibody against the nucleocapsid protein of the target analyte. The capture molecule (24) comprises 0.75-1 μg of the peptide or 0.75-1 μg of the antibody.

The present invention provides a lateral flow assay device (100) where the control region (26) comprises 0.5 μg to 1 μg of the spike protein or the nucleocapsid protein of the target analyte.

The present invention provides a lateral flow assay device (100) where the target analyte is an enveloped virus selected from SARS CoV1, SARS CoV2, MERS CoV, influenza virus, Hepatitis B and C, and Ebola virus.

The present invention provides a lateral flow assay device (100) comprising a porous membrane (20) mounted on a solid support (10). The said porous membrane have a sample pad (14) for receiving a liquid sample (12) comprising a SARS CoV2 virus at a first end and an absorbent pad (28) at the second end. The said solid support (10) permits capillary flow of the liquid sample comprising the target analyte from the sample pad (14) to the absorbent pad (28). It is characterized in that the porous membrane (20) comprises a conjugate pad (16) comprising gold nanoparticle sensor conjugate (18). The said conjugate comprises gold nanoparticle have a particle size of 10 nm to 20 nm conjugated with a peptide capable of binding with the S1 spike protein of SARS CoV2 and has a SEQ ID NO: 1 or said conjugate comprises gold nanoparticle have a particle size of 10 nm to 20 nm conjugated with an anti-S1mAB or anti-NmAB of the SARS CoV2. The porous membrane (20) includes a test region (22) comprising an immobilized capture molecule (24) is provided. The said capture molecule is a peptide capable of binding with the S1 spike protein of SARS CoV2 and has a SEQ ID NO: 1 or is an anti-S1mAB or anti-NmAB of the SARS CoV2. Optionally a control region (26) comprising the S1 spike protein in the SARS CoV 2 virus immobilized on the porous membrane (20) is provided.

The present invention relates to a lateral flow assay method for detecting a target analyte in a sample comprising applying a sample (12) containing the target analyte on the sample pad (14) of the device (100) of the present invention. The sample is allowed to flow from the sample pad (14) to the test region (22) through the conjugate pad (16). The presence or absence of the target analyte in the test region (22) is detected with a change in colour from red to purple in about 60 seconds to 300 seconds.

The method of the present invention further comprises allowing the sample flow further to the control region (26). The colour change from red to purple is observed in the control region in about 60 seconds to about 300 seconds. The colour change confirms the presence or absence of the target analyte in the test region (22).

The present invention provides a method where the sample (12) is oral swab, nasal swab, sputum, or saliva.

The present invention provides a method where the sample (12) is diluted in a buffer selected from phosphate buffered saline.

The present invention provides a method where the sample the target analyte is an enveloped virus selected from SARS CoV 1, SARS CoV2, MERS CoV, influenza virus, Hepatitis B and C, and Ebola virus.

The present invention provides a method for detecting analyte in a sample where said analyte is a SARS CoV-2. The said method comprising applying a sample (12) containing the target analyte on the sample pad (14) of the device (100) of the present invention. The sample is allowed the sample to flow from the sample pad(14) to the test region (22) through the conjugate pad (16). The presence of the SARS CoV2 virus in the test region is detected with a change in colour from red to purple in about 60 seconds to about 300 seconds.

The method of the present invention further comprises allowing the sample flow further to the control region (26). The colour change from red to purple is observed in the control region in about 60 seconds to about 300 seconds; and confirms the presence of the SARS CoV 2 virus in the test region.

The method of the present invention detects the presence of the SARS CoV 2 virus in the test region with a change in colour from red to purple in about 60 seconds to about 180 seconds.

The method of the present invention detects SARS CoV2 virus up to 192TCID50.

The present invention provides a method where the detection has 90%-92% sensitivity and 98%-100% specificity for SARS CoV2 virus.

The present invention provides a kit for detecting SARS CoV2 virus in a sample comprising a lateral flow assay device (100) of the present invention and dilution buffer selected from phosphate buffer saline.

The present invention provides a method for quantitatively detecting a target analyte in a sample comprising the step of measuring absorbance of gold nanoparticles having particle size of 10 nm-20 nm conjugated with a peptide that specifically binds with a protein in the target analyte or of said gold nanoparticle having particle size of 10 nm-20 nm conjugated with an antibody against the protein in the target analyte at 525 nm. A 50-200 μl of the sample is mixed with 50-100 μl said gold nanoparticles, change in colour from red to purple is observed and the absorbance is measured at 700 nm. The absorbance ratio of the 525 nm and the 700 nm is calculated where the absorbance ratio of 525 nm to 700 nm is inversely proportional to the amount of target analyte in the sample.

The present invention provides a method for quantitatively detecting a target analyte, where the target analyte is an enveloped virus selected from SARS CoV1, SARS CoV2, MERS CoV, influenza virus, Hepatitis B and C, and Ebola virus.

The present invention provides a method for quantitatively detecting a SARS CoV 2 in a sample comprising the step of measuring absorbance of gold nanoparticles having particle size of 10 nm-20 nm conjugated with a peptide is capable of binding with the S1 spike protein of SARS CoV2 and has a SEQ ID NO: 1 or of said gold nanoparticle having particle size of 10 nm-20 nm is conjugated with an anti-S1mAB or anti-NmAB of the SARS CoV2 at 525 nm. A 50-200 μl of the sample is mixed with 50-100 μl said gold nanoparticles, change in colour from red to purple is observed and the absorbance is measured at 700 nm. The absorbance ratio of the 525 nm and the 700 nm is calculated where the absorbance ratio of 525 nm to 700 nm is inversely proportional to the amount of SARS CoV2 in the sample.

The present invention provides a method where the absorbance ratio of 525 nm to 700 nm is 1.4 to 7.6.

The present invention provides a conjugate comprising 10 nm to 20 nm gold nanoparticle and a peptide is capable of binding with the S1 spike protein of SARS CoV2 and has a SEQ ID NO: 1.

The present invention provides a method of diagnosing COVID-19 in a patient sample comprising diluting the sample in a buffer. The diluted sample is applied to the lateral flow assay device (100) of the present invention. The change in colour from red to purple from about 60 seconds to about 300 seconds indicates the presence of SARS CoV2 virus in the sample. The change in colour from red to purple is within 60 to 180 seconds. The sample is from symptomatic or asymptomatic patient. The sample is oral swab, nasal swab, sputum, or saliva.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

Reference will be made to embodiments of the invention, examples of which may be illustrated in the accompanying figures. These figures are intended to be illustrative, not limiting. Although the invention is generally described in the context of these embodiments, it is understood that it is not intended to limit the scope of the invention to these particular embodiments:

FIG. 1 is a schematic diagram of a lateral flow assay device, according to an embodiment of the present invention.

FIG. 2 is a schematic diagram illustrating the gold nanoparticle conjugate sensor molecules according to an embodiment of the present invention, binding to an analyte showing colour change.

FIG. 3 illustrates the lateral flow assay method according to an embodiment of the present invention with virus particle size of 10\hat{ }3 to 10\hat{ }6.

FIG. 4 illustrates the lateral flow assay method according to an embodiment of the present invention with spot development in the control region with the conjugate gold nanoparticle of the present invention and the spike protein S1 in SARS CoV-2 virus.

FIG. 5 illustrates the sensitivity and specificity of detection of SARS-CoV-2, according to an embodiment of the present invention.

FIG. 6 illustrates the results of quantitative assessment of gold nanoparticles biosensors, according to an embodiment of the present invention.

### DETAILED DESCRIPTION OF THE INVENTION

The present invention relates biosensors (device) and bioassays (method) and more particularly to nanoparticle conjugate biosensors. The device and the method of the present invention is simple to perform and is an inexpensive test for analytes such as pathogens or other biomarkers by having nanoparticle conjugate biosensors with the capability to bind at multiple sites or regions in the target analyte. This multisite binding of the biosensors is achieved by small sized sensors that make the detection easier, faster, simpler, sensitive and specific.

The present invention provides small sized nanoparticles conjugate biosensors such as nanoparticles bound to a peptide or an antibody, wherein these peptides or antibodies may selectively bind to a target analyte at multiple sites, thus resulting in aggregation of nanoparticles. A colour change indicates the detection of presence or absence of the target analyte. The property of aggregation and colour change can be observed in small gold nanoparticles which are of size range from 10 to 20 nm, preferably from 10 to 15 nm, more preferably 10 nm. When a sensor such as peptide or antibody, preferably a peptide, binds close to each other on the analyte to be detected, even without aggregation, the colour change happens from red to purple. In other words, small multi epitope binding peptides or antibodies conjugated to small size gold nanoparticles bind close to each other on the analyte similar to aggregation resulting in colour development and change from red to purple.

In one embodiment, the present invention relates to a lateral flow assay device. The present invention relates to a lateral flow assay device (100) comprising porous membrane (20) mounted on a solid support (10). The said porous membrane can have a sample pad (14) for receiving a liquid sample (12) comprising a target analyte at a first end and an absorbent pad (28) at the second end. The said solid support (10) permits capillary flow of the liquid sample comprising the target analyte from the sample pad (14) to the absorbent pad (28). It is characterized in that the porous membrane (20) can comprise a conjugate pad (16) comprising gold nanoparticle sensor conjugate (18). The said gold nanoparticle sensor conjugate comprises gold nanoparticle having a particle size of 10 nm to 20 nm conjugated with a peptide that specifically binds with a protein in the target analyte or said gold nanoparticle sensor conjugate comprises gold nanoparticle having a particle size of 10 nm to 20 nm conjugated with an antibody against the protein in the target analyte. The porous membrane (20) comprises a test region (22) with an immobilized capture molecule (24) can be provided. The said capture molecule is a peptide capable of specifically binding to the protein in the target analyte or an antibody against protein in the target analyte. Optionally, the porous membrane (20) can have a control region (26) with the protein in the target analyte immobilized on the porous membrane (20) can be provided.

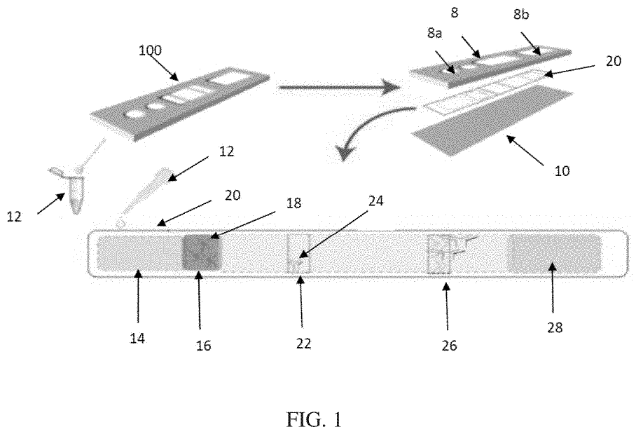

FIG. 1 illustrates a schematic diagram of lateral flow assay device according to an embodiment of the present invention. The porous membrane (20) can be mounted on a solid support (10). The porous membrane can be nitrocellulose membrane or polyvinylidene fluoride (PVDF) membrane. The porous membrane (20) can have a sample pad (14) at the first end for receiving a liquid sample (12) comprising a target analyte. The sample pad can be of material with cellulose acetate or glass fiber. Preferably, the sample pad can be soaked in 5% BSA and dried completely before using it in the assay. The porous membrane (20) can have an absorbent pad (28) at the second end for absorbing excess fluid that flows through the membrane. The solid support (10) permits capillary flow of the liquid sample comprising the target analyte from the sample pad (14) to the absorbent pad (28).

The porous membrane (20) comprises a conjugate pad (16) comprising gold nanoparticle sensor conjugate (18). The said conjugate can have gold nanoparticle with a particle size of 10 nm to 20 nm gold nanoparticle. Preferably, the nanoparticle can have a particle size of 10 nm to 15 nm, more preferably 10 nm. In one embodiment, the gold nanoparticles having a size of 10 nm to 15 nm, more preferably 10 nm can be conjugated with a peptide (sensor/biosensor) that specifically binds with a protein in the target analyte. The gold nanoparticle conjugate can comprise 30 μg-50 μg of the peptide. In another embodiment, the gold nanoparticle having a size of 10 nm to 15 nm, more preferably 10 nm can be conjugated with a monoclonal antibody (sensor/biosensor) against the protein in the target analyte. The gold nanoparticle conjugate can then comprise 0.5 μg-1 μg of the antibody. The conjugate pad can be made of glass fiber filters, cellulose filters, surface-treated (hydrophilic) polyester or propylene fibers.

The gold nanoparticle conjugate comprising the peptide or antibody can be prepared by spinning down the 10 nm diameter gold nanoparticles at 21000 g or 15000 rpm for 1 hour. The supernatant can be removed and precipitate can be resuspended in 2 mM Sodium tetraborate decahydrate. The peptide or antibody can be dissolved in distilled water or in Tris 50 mM buffer pH7 at a desired concentration and incubated at 25° C. mixing at 750 RPM for 1 hour. This mixture can be then combined with a required amount of 10% Bovine Serum Albumin

(BSA) made in 2 mM Sodium tetraborate decahydrate to make it to final concentration of 1% BSA and incubated at 25° C. mixing at 750 rpm for 1 hour. This final mixture can be centrifuged at 21000 g or 15000 RPM for 1 hour. After centrifugation, the supernatant can be removed, and the precipitate can be resuspended in 2 mM Sodium tetraborate decahydrate for coating the conjugate pad. The conjugate can be precoated on a conjugate pad of a lateral flow assay device (100).

The target analyte can be a virus, particularly an enveloped virus selected from SARS CoV1, SARS CoV2, MERS CoV, influenza virus, Hepatitis B and C, and Ebola virus. When the target analyte is an enveloped virus, the gold nanoparticle can be conjugated preferably with the peptide capable of specifically binding with a spike protein or another protein of the enveloped virus. The another protein can be a membrane protein. The gold nanoparticle can preferably be conjugated with the antibody against the nucleocapsid protein of the virus.

In an exemplary embodiment, the target analyte is SARS CoV2 virus. The gold nanoparticle conjugate for detecting SARS CoV2 virus can comprise of gold nanoparticle having a size of 10 nm to 15 nm, more preferably 10 nm conjugated with a peptide of SEQ ID NO:1 against the spike S1 protein. The gold nanoparticle conjugate can comprise 30 μg-50 μg of the peptide of SEQ ID NO:1. In another embodiment, the gold nanoparticle conjugate for detecting SARS CoV2 virus can comprise gold nanoparticle having a size of 10 nm to 15 nm, more preferably 10 nm conjugated with an anti-S1mAB or anti-NmAB of the SARS CoV2.The gold nanoparticle conjugate can then comprise 0.5 μg-1 μg of the antibody.

In a preferred embodiment, the present invention provides a conjugate comprising 10 nm to 20 nm gold nanoparticle and a peptide is capable of binding with the S1 spike protein of SARS CoV2 and has a SEQ ID NO: 1. The gold nanoparticle can preferably have a size of 10 nm to 15 nm, more preferably 10 nm. The conjugate of the present invention can be precoated on a conjugate pad of a lateral flow assay device (100).

The porous membrane (20) comprises a test region (22) comprising an immobilized capture molecule (24). In one embodiment, the capture molecule can be a peptide (capture peptide) capable of specifically binding to the protein in the target analyte. In another embodiment, the capture molecule can be an antibody (capture antibody) against protein in the target analyte. The capture peptide or capture antibody can be immobilized by methods known in the art. In one embodiment, when the lateral flow assay device comprises gold nanoparticle conjugate with a peptide (sensor/biosensor) that specifically binds with a protein in the target analyte, the capture molecule can be a capture antibody. In another embodiment, when the gold nanoparticle conjugate in the lateral flow assay device is gold nanoparticle conjugated with an antibody (sensor/biosensor) against the protein in the target analyte, the capture molecule can be a capture peptide. In the preferred embodiment, the nanoparticle can be conjugated with the peptide and the capture molecule can be an antibody against protein in the target analyte.

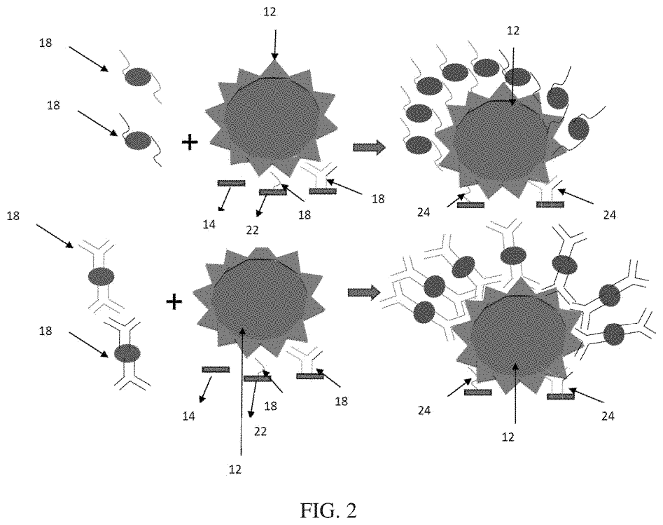

FIG. 2 illustrates the sensor molecules binding to an analyte resulting in colour change. FIG. 2 shows that diluted sample containing the target analyte (12) is applied on to the sample pad (14) following which the analyte binds to the gold nanoparticle biosensor, peptide or antibody (18) which is in red colour. The analyte bound with the nanoparticle conjugate flows through capillary action to the test region (22) and is captured by capture peptide or antibody (24) on porous membrane changing the red colour to a purple colour line. The gold nanoparticles are 10 nm in size and when they are near to each other due to binding of peptide/antibody sensor on the analyte represents aggregation of conjugated gold nanoparticles on analyte thus changing the colour from red to purple.

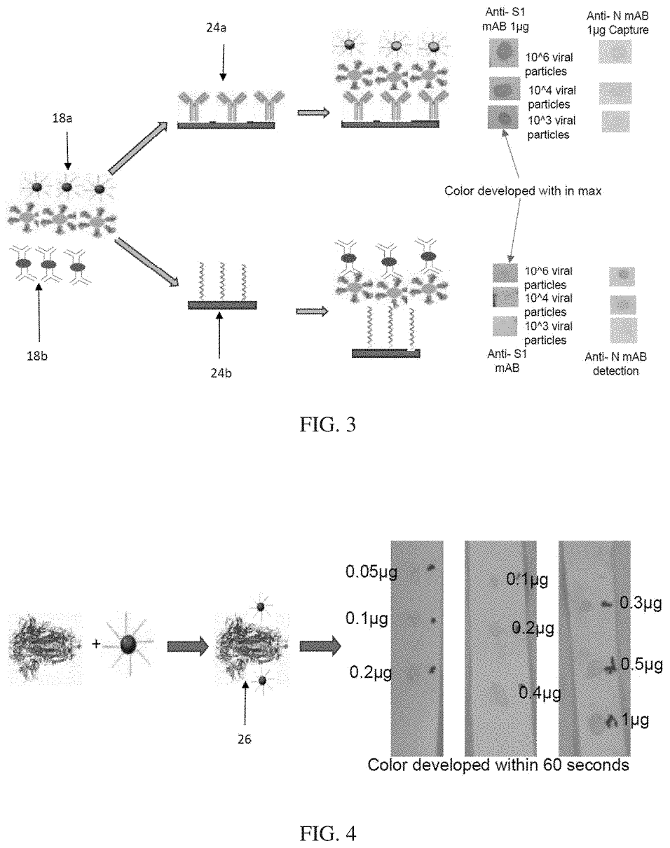

In an exemplary embodiment, the test region (22) comprises an immobilized capture molecule (24); said capture molecule is a peptide capable of binding with the S1 spike protein of SARS CoV2 and has a SEQ ID NO: 1 or said gold nanoparticle is conjugated with an anti-S1mAB or anti-NmAB of the SARS CoV2. FIG. 3 illustrates results associated for test region (22) from the assay where anti-S1 monoclonal antibody or anti-N monoclonal antibody (18b) were spotted for capture(24b), in another set peptide (18a) was spotted for capture (24a). Virus containing samples were mixed with gold nanoparticle biosensor (peptide 4 or anti-S1 monoclonal antibody or anti-N monoclonal antibody) and blotted on the membranes for detection. FIG. 3 shows change in red colour to purple colour development in both the cases.

The lateral flow assay device of the present invention can optionally comprise a control region (26) comprising the protein in the target analyte immobilized on the porous membrane (20). The protein can be a spike protein or a membrane protein. Preferably, the protein immobilized on the control region can be a spike protein. The control region (26) preferably comprises 0.5 μg to 1 μg of the spike protein or the nucleocapsid protein of the target analyte. In one embodiment, sample and a control may be applied separately to a membrane. The conjugate of the present invention may be added dropwise after the sample or specimen binds to the membrane. If the specific analyte of interest is present in the specimen , the sensor conjugate will bind to it, thereby inducing a colour change for example from red to purple. The colour change of the sample in the test region may be compared to the colour change of the control to minimize error or to determine relative concentration.

In an exemplary embodiment, the present invention provides a lateral flow assay device (100) for detecting SARS CoV2 virus. The device according to the embodiment comprises a porous membrane (20) mounted on a solid support (10). The said porous membrane have a sample pad (14) for receiving a liquid sample (12) comprising a SARS CoV2 virus at a first end and an absorbent pad (28) at the second end. The said solid support (10) permits capillary flow of the liquid sample comprising the target analyte from the sample pad (14) to the absorbent pad (28). It is characterized in that the porous membrane (20) comprises a conjugate pad (16) comprising gold nanoparticle sensor conjugate (18). The said gold nanoparticle sensor conjugate have particle size of 10 nm to 20 nm conjugated with a peptide is capable of binding with the S1 spike protein of SARS CoV2 and has a SEQ ID NO: 1 or said gold nanoparticle sensor conjugate have particle size of 10 nm to 20 nm is conjugated with an anti-S1mAB or anti-NmAB of the SARS CoV2. A test region (22) comprising an immobilized capture molecule (24) is provided. The said capture molecule is a peptide capable of binding with the S1 spike protein of SARS CoV2 and has a SEQ ID NO: 1 or said gold nanoparticle is conjugated with an anti-S1mAB or anti-NmAB of the SARS CoV2. Optionally a control region (26) comprising the S1 spike protein in the SARS CoV 2 virus immobilized on the porous membrane (20) is provided. The S1 or N spotted to be detected or bind by B-AuNP can be as positive control at control region.

The device according to the present invention can have an outer cover (8) enclosing the membrane (20) and the solid support (10)with a display (8b) for the test region (22) and control region (26) and a slot or opening (8a) for applying sample on the sample pad (14).

In one embodiment, the present invention provides a lateral flow assay method for detecting an analyte in a sample. The method comprises applying a sample (12) containing the target analyte on the sample pad (14) of the device (100). The sample is allowed to flow from the sample pad (14) to the test region (22) through the conjugate pad (16). The presence or absence of the target analyte in the test region (22) is detected with a change in colour from red to purple in about 60 seconds to 300 seconds.

The method of the present invention further comprises allowing the sample flow further to the control region (26); observing the colour change from red to purple in the control region in about 60 seconds to about 300 seconds; and confirming the presence or absence of the target analyte in the test region (22).

The sample (12) can be oral swab, nasal swab, sputum or saliva. The sample (12) can be diluted in a buffer selected from phosphate buffered saline. The buffer can be phosphate buffer saline with Tween 20 of molarity 1M, taken at a volume of 0.05-0.01%. The volume of sample can be 400 μl-500 μl. The target analyte can be an enveloped virus selected from SARS CoV1, SARS CoV2, MERS CoV, influenza virus, Hepatitis B and C, and Ebola virus. The sample pad (14) can be soaked in 5% BSA for 1 minute and dried at 37° C. until it is completely dried before applying the sample (12).

In an exemplary embodiment the present invention provides a method for detecting analyte in a sample (12) wherein said analyte is a SARS CoV-2. The said method comprises applying a sample (12) containing the target analyte on the sample pad (14) of the device (100) of the present invention. The sample is allowed to flow from the sample pad (14) to the test region (22) through the conjugate pad (16). The presence of the SARS CoV2 virus is detected in the test region with a change in colour from red to purple in about 60 seconds to about 300 seconds. FIG. 4 illustrates the results of control region or spot development where recombinant spike S1 protein was spotted in different concentrations and peptide conjugated gold nanoparticles were used to detect and as seen in the results, very low concentrations of S1 protein were detected.

The method of the present invention further comprises allowing the sample to flow further to the control region (26); observing the colour change from red to purple in the control region in about 60 seconds to about 300 seconds; and confirming the presence of the SARS CoV 2 virus in the test region. Preferably, the change can be detected in about 60 seconds to about 180 seconds.

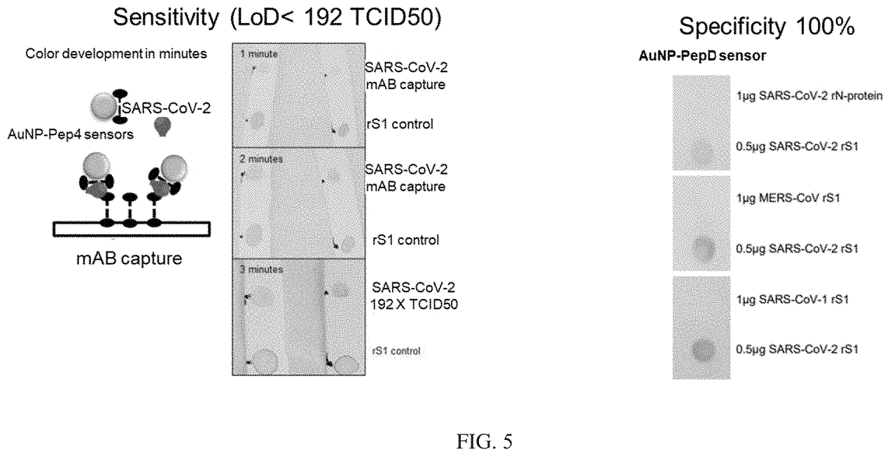

The method according to the present invention can detect SARS CoV2 virus up to 192TCID50. FIG. 5 illustrates the limit of detection of SARS-nCoV-2 virus up to 192 TCID50 (Median Tissue Culture Infectious Dose). The method of detecting SARS CoV2 according to the present invention shows 90%-92% sensitivity and 98%-100% specificity for SARS CoV2 virus. FIG. 5 shows that the assay is very specific where the gold nanoparticle sensor and capture antibody of the present invention does not bind to spike proteins of MERS (Middle East Respiratory Syndrome) virus or SARS-CoV1 (Severe Acute Respiratory Syndrome Coronavirus 1).

In another embodiment, the method can be performed by depositing a drop of on a solid surface attached with the sample. The appearance of the purple coloured spot on said solid surface indicates detection. In yet another embodiment, a multi-well plate having a plurality of wells may be used wherein each well holds a composition of nanoparticles bound to a detection peptide or molecule capable of detecting the analyte in a sample added to the wells. In some embodiments, the sensor may indicate the presence of an analyte by a visible colour change. In some instances, an additional reagent may be provided to aid colour change. For example, hydrochloric acid or sulfuric acid may be added in conjunction with iron oxide nanoparticles.

In another embodiment, the method can be performed by adding the sample containing the target analyte to a test tube followed by addition of the said sensor conjugate. A color change indicates detection of the analyte in the sample. In some embodiment, two separate tubes may be filled with a small volume of said sensor conjugate. The sample may be added to one tube and a control , for example, a positive control, may be added to the other tube. The said sensor conjugate binds to the analyte in control. If the analyte is present in the sample, the color of the sample will change, thus indicating detection of the analyte. The color change of the sample may be compared to the color change of the control to minimize error or to determine relative concentration. Alternatively, the sample may be added to a first tube and the control may be added to a second tube. The sensor conjugate may subsequently be added to both tubes. A color change indicates the presence of the analyte. The color change of the sample may be compared to the color change of the control to minimize error or to determine relative concentration.

In one embodiment, the present invention provides a kit for detecting SARS CoV2 virus in a sample. The kit can comprise a lateral flow assay device (100) of the present invention and dilution buffer selected from phosphate buffer saline. The kit can further comprise a tube or vial for collecting a sample. The sample can be oral swab, nasal swab, sputum or saliva. The kit can comprise a swab.

A sample collection vial or tube with dilution buffer (phosphate buffer saline) is used for collection of saliva, a swab for nasal or oral sample is used to collect sample and put into dilution buffer. This vial or tube has a dropper cap using which a few drops of diluted sample is applied on to the sample pad following which the analyte or SARS-nCoV-2 virus in this case binds to the gold nanoparticle biosensor (peptide or antibody) and is further captured by capture peptide or antibody on porous membrane for detection by initial red colour development and then change to purple.

The reading of the kit or method of the present invention is simple as it can clearly show if the binding occurred or not. As the binding happens, there is a development of red colour and also change to purple. The analyte or SARS-nCoV-2 detection kit or method described herein is extremely user friendly since it involves few steps, which are simple for any user to perform.

In one embodiment, the present invention provides a method for quantitatively detecting a target analyte in a sample comprising the step of measuring absorbance of gold nanoparticles having particle size of 10 nm-20 nm conjugated with a peptide that specifically binds with a protein in the target analyte or said gold nanoparticle is conjugated with an antibody against the protein in the target analyte at 525 nm; mixing 50-200 μl of the sample with 50-100 μl said gold nanoparticles, observing the change in colour from red to purple; and measuring the absorbance at 700 nm; and calculating the absorbance ratio of the 525 nm and the 700 nm; wherein the absorbance ratio of 525 nm to 700 nm is inversely proportional to the amount of target analyte in the sample.

The target analyte can be an enveloped virus selected from SARS CoV 1, SARS CoV2, MERS CoV, influenza virus, Hepatitis B and C, and Ebola virus.

The sample of any time such as saliva, nasal swab or oral swab were mixed with 4000 dilution buffer (phosphate buffer saline) followed by mixing 50-2000 of this diluted sample with gold nanoparticle peptide sensor conjugate (100 μl). The colour change from red to purple is observed by measuring absorbance before and after sample addition at 525 nm and 700 nm. It is known that absorbance at 700 nm increases with aggregation of gold nanoparticles and in this case the gold nanoparticles peptide biosensor binds near to each other on the virus thus resembling the aggregation of gold nanoparticles this absorbance at 700 nm increases resulting in reducing the value of absorbance ratio at 525 nm/700 nm. The increase in the viral load resulted in reduction of ratio of 525 nm/700 nm.

In an exemplary embodiment, the present invention provides a method for quantitatively detecting a SARS CoV 2 in a sample comprising the step of measuring absorbance of gold nanoparticles having particle size of 10 nm-20 nm conjugated with a peptide is capable of binding with the S1 spike protein of SARS CoV2 and has a SEQ ID NO: 1 or said gold nanoparticle is conjugated with an anti-S1mAB or anti-NmAB of the SARS CoV2 at 525 nm; mixing 50-200 μl of the sample with 50-100 μl of said gold nanoparticles, observing the change in colour from red to purple; and measuring the absorbance at 700 nm; and calculating the absorbance ratio of the 525 nm and the 700 nm; wherein the absorbance ratio of 525 nm to 700 nm is inversely proportional to the amount of target analyte in the sample.

The sample can be oral swab, nasal swab, sputum or saliva. The sample can be added to 100 μl of gold nanoparticle conjugate and analyte, or biomarker can be detected using spectrometric absorbance measurement at 525 nm and 700 nm. Few of the advantages of the present invention are that complete method can be performed in less than 5 minutes, is highly specific and sensitive.

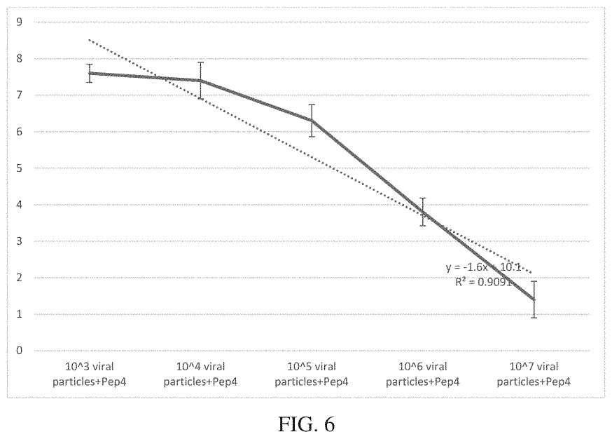

FIG. 6 illustrates the results of quantitative assessment of gold nanoparticles biosensors detecting different loads of SARS-nCoV-2 viral particles by measuring the ratio of absorbance at 525 nm and 700 nm. With increased viral load, the ratio decreases as absorbance at 700 nm increases. The absorbance ratio of 525 nm to 700 nm can be in a range of 1.4 to 7.6.

In an embodiment, gold nanoparticle peptide sensor conjugate of the present invention can be mixed with a sample. The colour change from red to purple is observed by measuring absorbance before and after sample addition at 525 nm and 700 nm. The sample can be oral swab, nasal swab, sputum, or saliva.

In one embodiment, the present invention provides a method of diagnosing COVID-19 in a patient sample comprising diluting the sample in a buffer, applying the diluted sample to the lateral flow assay device (100) of the present invention; observing change in colour from red to purple from about 60 seconds to about 300 seconds indicates the presence of SARS CoV2 virus in the sample. Preferably, the change in colour from red to purple is within 60 to 180 seconds.

The sample can be from symptomatic or asymptomatic patient. The sample can be oral swab, nasal swab, sputum, or saliva.

### EXAMPLES

The spike monoclonal antibody is from MP biomedicals catalogue number 0720302. N antibody is from Pentavalent private limited, catalogue number is pvbsp20102. Spike protein is from Sinobiologicals Inc catalogue number 40591-v08b1.

Bovine serum albumin-Sigma-Catalogue number: A3294-100; Gold nanoparticles 10 nm-Sigma-catalogue number: 741957-100m1; Sodium tetraborate decahydrate-Sigma-catalogue number: S9640-2.5KG; Glass fiber sheet (Conjugate pad)-Axivia; Sample pad (Cellulose membrane)-Axivia; Nitrocellulose membrane-Axivia; Absorbent pad-Axivia; Phosphate buffer saline tablets-Sigma-catalogue number: P4417-100TA; Sucrose-Sigma-Catalogue number: S0389-1KG

With regards to the patient's sample saliva or nasal swab or oral swab are seen to be working and specifically in experiments saliva was used. The patient samples are collected from NITTE medical university, dalerikata, Mangalore, Karnataka.

### Example 1: Preparing Gold Nanoparticles Peptide Conjugate

The gold nanoparticles of 10 nm size were purchased in Sodium citrate buffer and then centrifuged at 21000 g or 15000 rpm for 1 hour. After centrifugation, the supernatant was removed and 2 mM Sodium tetraborate decahydrate was added and mixed well following which the mixture was centrifuged again at 21000 g or 15000 rpm for 1 hour. After centrifugation, the supernatant was removed and fresh 2 mM Sodium tetraborate decahydrate buffer was added along with 30 μg to 60 μg of peptide sensor per 400 μl of gold nanoparticles of size 10 nm following which the mixture was incubated at 25° C. with constant mixing at 750 rpm for 1 hour. After 1 hour 2% BSA(10% conc.) was added to make final BSA concentration to 1% and the mixture was incubated at 25° C. with constant mixing at 750 rpm for 1 hour. Following BSA blocking, the mixture was centrifuged at 21000 g or 15000 rpm for 1 hour and supernatant was removed and resuspended in 10 times less volume of 2 mM Sodium tetraborate decahydrate than the initial volume of gold nanoparticles. This final solution is used for sensing the analyte in a sample.

The peptide used in the example is SEQ ID NO.1: ALHLYSAEQKQM

### Example 2: Preparing Gold Nanoparticles Antibody Conjugate

The gold nanoparticles of 10 nm size were purchased in Sodium citrate buffer and then centrifuged at 21000 g or 15000 rpm for 1 hour. After centrifugation, the supernatant was removed and 2 mM Sodium tetraborate decahydrate was added and mixed well following which the mixture was centrifuged again at 21000 g or 15000 rpm for 1 hour. After centrifugation, the supernatant was removed and fresh 2 mM Sodium tetraborate decahydrate buffer was added along with 0.5 μg-1 μg of antibody per 1 ml of gold nanoparticles of 10 nm size following which the mixture was incubated at 25° C. with constant mixing at 750 rpm for 1 hour. After 1 hour 2% BSA(10% conc.) was added to make final BSA concentration to 1% and the mixture was incubated at 25° C. with constant mixing at 750 rpm for 1 hour. Following

BSA blocking, the mixture was centrifuged at 21000 g for 1 hour and supernatant was removed and resuspended in 10 times less volume of 2 mM Sodium tetraborate decahydrate than the initial volume of gold nanoparticles. This final solution is used for sensing the analyte in a sample.

### Example 3: Spotting Capture Peptide or Antibody on Membrane

In order to capture the analyte to be detected specifically a 0.75-1 μg of the monoclonal antibody or 0.75-1 μg of the peptide was used as capture molecule. In this case, peptide which specifically binds to S1 protein or anti-S1 mAB or anti-N mAB were mixed in distilled water solution of 2% sucrose and 2% trehalose making final concentration of sucrose and trehalose 1% each. This biosensor (peptide, or mABs) mixture with sucrose or trehalose is spotted on PVDF or nitrocellulose membrane and dried at 37° C. for 2 hours before blocking the remaining surface with 1% BSA for 15 minutes following which the membrane is dried again at 37° C. for 2 hours before using for assay.

The peptide used in the example is SEQ ID NO.1: ALHLYSAEQKQM

### Example 4: Spotting Control Protein with Sucrose or Trehalose

In order to make sure the gold nanoparticles biosensor is working a control spot or line of corresponding protein was to be spotted on the PVDF or nitrocellulose membrane. In this case, 1 μg or 0.5 μg of S1 protein mixed with 2% sucrose and trehalose to give final 1% sucrose and trehalose concentration. This final mixture is spotted on the PVDF or nitrocellulose membrane and dried for 2 hours following which the remaining membrane is blocked with 1% BSA for 15 minutes and dried at 37° C. before using for assay.

### Example 5: Pretreatment of Sample Pad

The sample pad is soaked in 5% BSA for 1 minute and dried at 37° C. until it is completely dried before using it in the assay.

### Example 6: Binding the Gold Nanoparticles Biosensor on Conjugate Pad Glass Fiber

The gold nanoparticles once conjugated with biosensors, are applied to glass fiber conjugation pad. Once applied the conjugation pad is allowed to dry at room temperature till it is completely dry before use.

### Example 7: Lateral Flow Assay

The lateral flow assay is performed with sample being applied to sample pad which when goes forward binds to the gold nanoparticles biosensor conjugate and flows in order for the specific analyte to be captured by capture peptide or antibody and in doing so the colour develops to red and then changes to purple. Next the gold nanoparticle biosensor binds to control protein to give colour development.

### Example 8: Calorimetric Assay

The colorimetric assay consists of adding the 50-100 μl of sample diluted in dilution buffer to the tube or in a well of 96 well plate 100 μl of gold nanoparticle conjugate. Before and after the addition of sample to gold nanoparticle biosensor absorbance at 525 nm and 700 nm were taken to understand the binding of analyte to biosensor.

TABLE 1
525/700 nm absorbance ratio of gold nanoparticles

| Viral particles + | Ratio of | |
|---|---|---|
| Gold nanoparticles | absorbance at | Standard |
| biosensors | 525/700 nm | Error |
| 10\hat{ }3 viralparticles + Pep4 | 7.6 | 0.25 |
| 10\hat{ }4 viral particles + Pep4 | 7.4 | 0.5 |
| 10\hat{ }5 viral particles + Pep4 | 6.3 | 0.44 |
| 10\hat{ }6 viral particles + Pep4 | 3.8 | 0.38 |
| 10\hat{ }7 viral particles + Pep4 | 1.4 | 0.5 |

Table 1 shows the 525/700 nm absorbance ratio of gold nanoparticles along with increasing concentrations of SARS-nCoV2 virus. With increase in viral load, absorbance at 700 nm increases and ratio decreases.

### Example 9: Determination of Sensitivity and Specificity of Method for Detection of SARS CoV-2

To illustrate the sensitivity and specificity of our test kit, 20 SARS-nCoV-2 positive samples and 5 negative samples suffering from influenza were selected. Out of 20 SARS-nCoV-2 samples, 18 samples had a viral copy number more than 50 copies of viral RNA and 2 had less than 50 copies of viral RNA. A typical asymptomatic infected person has a viral load of more than 315 copies of viral RNA copies per millilitre of sample (1). Our invention (antigen detection in less than 3 minutes) identified 18 out of 18 samples with viral copy numbers above 50, but 2 samples below 50 copy number were not detected. This data clearly suggests that our invention can identify asymptomatic infected individuals with a sensitivity near to RT-PCR and better than any rapid antigen detection kit. The 5 influenza samples were negative in our test too.

The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the disclosure.

What is claimed is:

1. A lateral flow assay device (100) comprising:
a porous membrane (20) mounted on a solid support (10); said porous membrane having a sample pad (14) for receiving a liquid sample (12) comprising a target analyte at a first end and an absorbent pad (28) at a second end, said solid support permits capillary flow of the liquid sample comprising the target analyte from the sample pad (14) to the absorbent pad (28);
characterized in that:
a. the porous membrane (20) comprises a conjugate pad (16) comprising a gold nanoparticle sensor conjugate (18), said conjugate comprises gold nanoparticles having a particle size of 10 nm to 20 nm conjugated with a peptide that specifically binds with a protein in the target analyte or said conjugate comprises gold nanoparticles having a particle size of 10 nm to 20 nm conjugated with an antibody against the protein in the target analyte;
b. a test region (22) comprising an immobilized capture molecule (24); said capture molecule is a peptide capable of specifically binding to the protein in the target analyte or an antibody against the protein in the target analyte; and
c. optionally a control region (26) comprising the protein in the target analyte immobilized on the porous membrane (20).

2. The lateral flow assay device (100) as claimed in claim 1, wherein the gold nanoparticle is conjugated with the peptide capable of specifically binding with the protein in the liquid sample (12) and the capture molecule comprises the immobilized antibody against the protein in the liquid sample (12).

3. The lateral flow assay device (100) as claimed in claim 1, wherein the gold nanoparticle is conjugated with the antibody against the protein in the liquid sample (12) and the capture molecule comprises the peptide capable of specifically binding with the protein in the liquid sample (12).

4. The lateral flow assay device (100) as claimed in claim 1, wherein the gold nanoparticle is conjugated with the peptide capable of specifically binding with a spike protein or a protein of the target analyte or with the antibody against the nucleocapsid protein of the target analyte.

5. The lateral flow assay device (100) as claimed in claim 4, wherein the gold nanoparticles comprise 30 μg-50 μg of the peptide or 0.5 μg-1 μg of the antibody.

6. The lateral flow assay device (100) as claimed in claim 1, wherein the capture molecule (24) is the peptide capable of specifically binding with a spike protein or a protein of the target analyte or the antibody against the nucelocapsid protein of the target analyte.

7. The lateral flow assay device (100) as claimed in claim 1, wherein the capture molecule (24) comprises 0.75-1 μg of the peptide or 0.75-1 μg of the antibody.

8. The lateral flow assay device (100) as claimed in claim 1, wherein the control region (26) comprises 0.5 μg to 1 μg of the spike protein or the nucleocapsid protein of the target analyte.

9. The lateral flow assay device (100) as claimed in claim 1, wherein the target analyte is an enveloped virus selected from SARS CoV1, SARS CoV2, MERS CoV, influenza virus, Hepatitis B and C, and Ebola virus.

10. The lateral flow assay device (100) as claimed in claim 1, wherein the target analyte is a SARS CoV2 virus, the protein is a S1 spike protein of SARS CoV2 and has a SEQ ID NO: 1, and the antibody against the protein in the target analyte is an anti-S1mAB or anti-NmAB of the SARS CoV2.

11. A lateral flow assay method for detecting a target analyte in a sample comprising:
a. applying a sample (12) containing the target analyte on the sample pad (14) of the device (100) as claimed in claim 1;
b. allowing the sample to flow from the sample pad (14) to the test region (22) through the conjugate pad (16); and
c. detecting a presence or absence of the target analyte in the test region (22) with a change in color from red to purple in about 60 seconds to about 300 seconds.

12. The method as claimed in claim 11, further comprising:
allowing the sample to flow further to the control region (26);
observing the color change from red to purple in the control region in about 60 seconds to about 300 seconds; and
confirming the presence or absence of the target analyte in the test region (22).

13. The method as claimed in claim 11, wherein the sample (12) is an oral swab, a nasal swab, sputum or saliva.

14. The method as claimed in claim 11, wherein the sample (12) is diluted in a buffer selected from phosphate buffered saline.

15. The method as claimed in claim 11, wherein the target analyte is an enveloped virus selected from SARS CoV1, SARS CoV2, MERS CoV, influenza virus, Hepatitis B and C, and Ebola virus.

16. The method as claimed in claim 11, wherein said target analyte is a SARS CoV-2 virus, the protein is a S1 spike protein of SARS CoV2 and has a SEQ ID NO: 1, and the antibody against the protein in the target analyte is an anti-S1mAB or anti-NmAB of the SARS CoV2.

17. The method as claimed in claim 16, further comprising:
allowing the sample to flow further to the control region (26);
observing the color change from red to purple in the control region in about 60 seconds to about 300 seconds; and
confirming the presence of the SARS CoV-2 virus in the test region.

18. The method as claimed in claim 16, wherein detecting the presence of the SARS CoV-2 virus in the test region with a change in color from red to purple is in about 60 seconds to about 180 seconds.

19. The method as claimed in claim 16, wherein the method detects SARS CoV2 virus up to 192TCID50.

20. The method as claimed in claim 16, wherein the detection has 90%-92% sensitivity and 98%-100% specificity for SARS CoV2 virus.

21. A kit for detecting SARS CoV2 virus in a sample comprising:
a. a lateral flow assay device (100) as claimed in claim 10 and
b. dilution buffer selected from phosphate buffer saline.

22. A method for quantitatively detecting a target analyte in a sample comprising the steps of:
a. measuring absorbance of gold nanoparticles having a particle size of 10 nm-20 nm conjugated with a peptide that specifically binds with a protein in the target analyte or of said gold nanoparticle having a particle size of 10 nm-20 nm conjugated with an antibody against the protein in the target analyte at 525 nm;
b. mixing 50-200 μl of the sample with 50-1000 of said gold nanoparticles, observing the change in color from red to purple; and measuring the absorbance at 700 nm; and
c. calculating the absorbance ratio of the 525 nm and the 700 nm;
wherein the absorbance ratio of 525 nm to 700 nm is inversely proportional to an amount of target analyte in the sample.

23. The method as claimed in claim 22, wherein the target analyte is an enveloped virus selected from SARS CoV1, SARS CoV2, MERS CoV, influenza virus, Hepatitis B and C, and Ebola virus.

24. A method for quantitatively detecting a target analyte in a sample, wherein said analyte is a SARS CoV2 virus comprising the steps of:
a. measuring absorbance of gold nanoparticles having a particle size of 10 nm-20 nm conjugated with a peptide that is capable of binding with the S1 spike protein of SARS CoV2 and has a SEQ ID NO: 1 or of said gold nanoparticle having a particle size of 10 nm-20 nm conjugated with an anti-S1mAB or anti-NmAB of the SARS CoV2 at 525 nm;
b. mixing 50-200 μl of the sample with 50-1000 of said gold nanoparticles, observing a change in color from red to purple; and measuring the absorbance at 700 nm; and
c. calculating the absorbance ratio of the 525 nm and the 700 nm;
wherein the absorbance ratio of 525 nm to 700 nm is inversely proportional to an amount of SARS CoV2 in the sample.

25. The method as claimed in claim 24, wherein the absorbance ratio of 525 nm to 700 nm is 1.4 to 7.6.

26. A conjugate comprising 10 nm to 20 nm gold nanoparticle and a peptide that is capable of binding with the S1 spike protein of SARS CoV2 and has a SEQ ID NO: 1.

27. A method of diagnosing COVID-19 in a patient sample comprising:
a. diluting the sample in a buffer;
b. applying the diluted sample to the lateral flow assay device as claimed in claim 10;
c. a change in color from red to purple from about 60 seconds to about 300 seconds indicating the presence of SARS CoV2 virus in the sample.

28. The method as claimed in claim 27, wherein the change in color from red to purple is within 60 to 180 seconds.

29. The method as claimed in claim 28, wherein the sample is from a symptomatic or asymptomatic patient.

30. The method as claimed in claim 27, wherein the sample is an oral swab, a nasal swab, sputum or saliva.


SEQUENCE LISTING (1 sequence)

SEQ ID NO: 1 - protein, 12 aa, Artificial Sequence
  Peptide sequence
ALHLYSAEQKQM